(19) United States
(12) Patent Application Publication
(10) Pub. No.: US 2016/0032268 A1
(43) Pub. Date: Feb. 4, 2016

(54) UREASE PURIFICATION FROM JACK BEANS OR OTHER ORGANISMS

(71) Applicant: Medtronic, Inc., Minneapolis, MN (US)

(72) Inventors: Eric Grovender, Minneapolis, MN (US); Dianne L. Judd, Minneapolis, MN (US); Cindy Roberson, Plymouth, MN (US); Eric Hall, Plymouth, MN (US)

(73) Assignee: Medtronic, Inc., Minneapolis, MN (US)

(21) Appl. No.: 14/638,321

(22) Filed: Mar. 4, 2015

Related U.S. Application Data
(60) Provisional application No. 62/031,101, filed on Jul. 30, 2014.

(51) Int. Cl. C12N 9/80

(52) U.S. Cl. CPC C12N 9/80; C12Y 305/01005

(57) ABSTRACT

A method for the extraction and purification of urease from jack beans or other natural sources of urease. The method provides an efficient way to obtain purified urease from natural sources. The method can include defatting the natural sources of urease, extracting the urease from impurities, and further purification of the extracted urease.

### FIELD OF THE INVENTION

The invention relates to a method of isolating and purifying urease from jack beans or other organisms.

### BACKGROUND

Canavalia ensiformis (“jack beans”) is a common source of urea-degrading enzymes (“urease”). Other organisms that can serve as sources of urease include, but are not limited to soy beans, H. pylori bacteria, or other plants, bacteria or fungi (referred herein as also natural sources of urease). Urease has many uses, including use in sorbent dialysis to break down urea present in the spent dialysate into ammonium ions, which can be more easily removed by the other sorbent materials.

The vast majority of the mass of jack beans is made up of materials other than urease, including other proteins, fats and lipids. Known methods of isolating urease from jack beans are either inefficient, leading to waste of urease, or costly and time consuming. A method is therefore needed to efficiently extract the urease material from natural sources of urease.

Known methods of extracting and purifying urease from jack beans, such as the method of Sung, et al, Proc. Natl. Sci. Counc. B. ROC, Vol. 13, No. 4, 1989, pp. 250-257, suffer from low purity of the urease recovered. Sung describes an extraction from jack bean meal using 20% acetone after stirring at 20° C. for 5 minutes, followed by alkalifying the extract with heat treatment in the presence of 31-35% acetone, centrifuging the mixture, and then precipitating the urease by acidifying the extract. However, other proteins besides urease are present in the recovered material. Thus, an improved method of purifying the urease from jack beans or other natural sources of urease is needed.

### SUMMARY OF THE INVENTION

The first aspect of the invention is drawn to a method of isolating and purifying urease from natural sources of urease. In any embodiment of the first aspect of the invention, the method can comprise the steps of defatting the natural sources of urease with cold solvent to create defatted natural sources of urease, extracting the urease from the defatted natural sources of urease, and purifying the urease solution.

In any embodiment of the first aspect of the invention, the natural sources of urease can be jack beans.

In any embodiment of the first aspect of the invention, the step of defatting the natural sources of urease can comprise mixing the natural sources of urease with cold solvent to create a natural sources of urease/cold solvent mixture, and mechanically separating the natural sources of urease/cold solvent mixture.

In any embodiment of the first aspect of the invention, the hulls of the jack beans can be removed prior to the step of defatting the jack beans.

In any embodiment of the first aspect of the invention, the jack beans can be ground to make jack bean meal prior to the step of defatting the jack beans.

In any embodiment of the first aspect of the invention, the step of extracting the urease from the defatted natural sources of urease can comprise adding the defatted natural sources of urease to a buffer solution to create a natural sources of urease/buffer mixture, agitating the natural sources of urease/buffer mixture, and mechanically separating the natural sources of urease/buffer mixture, wherein the supernatant liquid contains the urease.

In any embodiment of the first aspect of the invention, the buffer solution can be a cold aqueous buffer.

In any embodiment of the first aspect of the invention, the step of purifying the urease solution can comprise using chromatography to purify the urease solution.

In any embodiment of the first aspect of the invention, the buffer solution can be ice cold.

In any embodiment of the first aspect of the invention, the chromatography can be size exclusion chromatography.

In any embodiment of the first aspect of the invention, the step of mixing the natural sources of urease with cold solvent can comprise agitating the natural sources of urease/cold solvent mixture by using a stir plate.

In any embodiment of the first aspect of the invention, the cold solvent can be cold acetone.

In any embodiment of the first aspect of the invention, the cold solvent can be ice cold solvent.

In any embodiment of the first aspect of the invention, the ice cold solvent can be kept at a temperature between −15° C. and 15° C., including between −10° C. and 15° C., between −5° C. and 15° C., between −0° C. and 15° C., between 5° C. and 15° C., 10° C. and 15° C., between −15° C. and 10° C., between −15° C. and 5° C., between −15° C. and 0° C., between −10° C. and 10° C., and between −5° C. and 5° C.

In any embodiment of the first aspect of the invention, the step of mixing the natural sources of urease with cold solvent can be carried out in an ice bath.

In any embodiment of the first aspect of the invention, the method can further comprise subjecting the purified urease to any one or both of a lyophilization process or spray-drying.

In any embodiment of the first aspect of the invention, the method can comprise the step of washing the natural sources of urease with hexane and/or heptane prior to the step of defatting the natural sources of urease.

In any embodiment of the first aspect of the invention, the step of purifying the urease can comprise the step of ultrafiltration.

The second aspect of the invention is drawn to a method of isolating and purifying urease from natural sources of urease. In any embodiment of the second aspect of the invention, the method of isolating and purifying urease from natural sources of urease can comprise the steps of extracting the urease from the natural sources of urease with acetone and buffer, precipitating out impurities from the extracted urease, mechanically separating the extracted urease solution to remove the precipitated impurities, and purifying the urease solution.

In any embodiment of the second aspect of the invention, the acetone and buffer can be ice cold.

In any embodiment of the second aspect of the invention, the step of precipitating out impurities from the extracted urease solution can comprise the steps of adding base to the extracted urease solution and heating the extracted urease solution.

In any embodiment of the second aspect of the invention, the step of purifying the urease can comprise removing the acetone and buffer from the urease, dissolving the remaining material in water to make an aqueous solution, and mechanically separating the aqueous solution to obtain a supernatant liquid wherein the urease is in the supernatant liquid.

In any embodiment of the second aspect of the invention, the step of purifying the urease solution can further comprise using chromatography to purify the urease solution.

In any embodiment of the second aspect of the invention, the chromatography used can be any of size exclusion chromatography, hydrophobic interaction chromatography or affinity chromatography.

In any embodiment of the second aspect of the invention, the acetone can be cold acetone.

In any embodiment of the second aspect of the invention, the method can further comprise the step of defatting the natural sources of urease prior to extracting the urease.

In any embodiment of the second aspect of the invention, the method can further comprise the step of washing the natural sources of urease with hexane and/or heptane prior to the step of defatting the natural sources of urease.

In any embodiment of the second aspect of the invention, the step of purifying the urease solution can comprise the step of ultrafiltration.

The third aspect of the invention is drawn to a method of purifying urease. In any embodiment of the third aspect of the invention, the method of purifying urease can comprise the step of modulating the pH of a liquid in the presence of urease.

In any embodiment of the third aspect of the invention, the method can further comprise mechanically separating fractions after modulating the pH of the liquid to isolate the fractions having an enhanced urease activity, wherein the urease activity of each fraction is defined on a per unit volume, dry mass, or protein mass basis.

In any of the methods of the present invention there is a proviso that the defatting step may be omitted.

### BRIEF DESCRIPTION OF THE DRAWINGS

FIG. 1 shows the agitation of a jack bean meal/acetone mixture on a stir plate.

FIG. 2 shows an overview of size exclusion chromatography.

FIG. 3 shows a flow chart of the extraction and purification of urease from jack beans according to one embodiment, with SDS-PAGE images.

FIG. 4 shows a SDS-PAGE image from the purification of the extracted urease.

FIG. 5 shows a flow chart of the extraction and purification of urease from jack beans according to another embodiment, with SDS-PAGE images.

FIG. 6 shows an SDS-PAGE image of the first 8 fractions collected during size exclusion chromatography of the purified urease.

FIG. 7 shows an SDS-PAGE image of the second 8 fractions collected during size exclusion chromatography of the purified urease.

FIG. 8 shows an SDS-PAGE image of the third set of 8 fractions collected during size exclusion chromatography of the purified urease.

FIG. 9 shows an SDS-PAGE image of the fourth set of 8 fractions collected during size exclusion chromatography of the purified urease.

FIG. 10 shows an SDS-PAGE image under reducing conditions of various steps in the method under different conditions.

FIG. 11 shows an SDS-PAGE image under non-reducing conditions of various steps in the method under different conditions.

### DETAILED DESCRIPTION OF THE INVENTION

Unless defined otherwise, all technical and scientific terms used herein generally have the same meaning as commonly understood by one of ordinary skill in the relevant art.

The articles “a” and “an” are used herein to refer to one or to more than one (i.e., to at least one) of the grammatical object of the article. By way of example, “an element” means one element or more than one element.

“Chromatography” refers to the process of separating a mixture by passing it in solution through a medium through which the components of the mixture move at different rates.

“Cold,” as used herein, refers to a temperature below 15° C., including below 10° C., 5° C., and 0° C.

The term “comprising” includes, but is not limited to, whatever follows the word “comprising.” Thus, use of the term indicates that the listed elements are required or mandatory but that other elements are optional and may or may not be present.

The term “consisting of” includes and is limited to whatever follows the phrase “consisting of.” Thus, the phrase indicates that the limited elements are required or mandatory and that no other elements may be present. The term “consisting essentially of” includes whatever follows the term “consisting essentially of” and additional elements, structures, acts or features that do not affect the basic operation of the apparatus, structure or method described

The terms “defat” or “defatting” refer to a process of removing lipids from a mixture of natural sources of urease as described herein including organic materials, generally containing ureases. Once the lipids have been removed, the material is “defatted.”

The term “hull,” as used in relation to seeds, refers to the dry outer covering of the seeds.

An “ice bath” is a container filled with a mixture of ice and liquid, often water, used to keep items placed within the ice bath at a low temperature.

The term “ice cold” refers to the temperature a substance will reach if it is left in an ice bath for a period of time. At standard pressure using a pure ice water bath, “ice cold” refers to a temperature of near 0° C. However, by using substances other than pure water for the ice bath, “ice cold” can refer to other temperatures above or below 0° C. It will be understood that “ice cold” refers to a temperature at or near the temperature of ice under certain conditions, and does not refer to a specific temperature.

“Jack beans” are the seeds of the plant canavalia ensiformis.

The term “jack bean meal” refers to jack beans that have been ground to fine particles.

“Lyophilization” refers to a process by which material is preserved by cooling the material to a temperature below the triple point of water and then reducing the pressure to cause the water present to sublimate.

“Mechanical separation” means any method of separating a liquid and a solid, including filtration, decanting, and centrifuging.

“Natural sources of urease,” as used herein, refers to any portion of a living or dead organism, including plants, bacteria, animal, or fungi from which urease may be extracted.

A “sensor” refers to a component capable of determining the states of one or more variables in a system.

“Size exclusion chromatography” refers to a process of separating a mixture by passing the mixture through a medium comprising hollow particles, causing the components of the mixture to move through the medium at different rates based on size.

A “supernatant” liquid is the liquid lying above a solid residue.

### Urease Extraction

The present invention relates to a method of isolating and purifying urease from jack beans or other organisms containing urease.

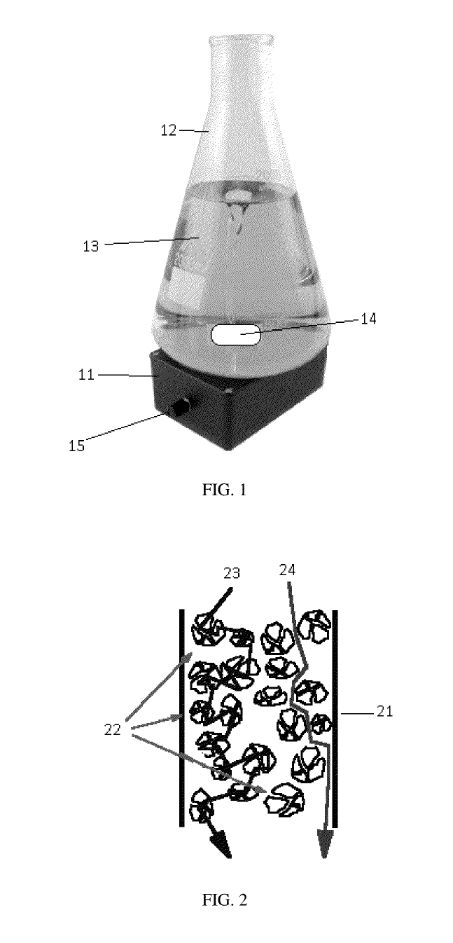

In any embodiment of the first, second or third aspects of the invention, the hull can be removed from the jack beans. The jack beans can then be ground into jack bean meal. In any embodiment of the first, second or third aspects of the invention, easily removable material can be removed from any natural source of urease. To remove the fat from the jack bean meal, the jack bean meal can be placed in cold acetone. In any embodiment of the first, second or third aspects of the invention, the acetone can be ice cold. In any embodiment of the first, second or third aspects of the invention, the acetone can be cooled to 0° C. In any embodiment of the first, second or third aspects of the invention, the acetone can be cooled to any temperature at or below 15° C. In any embodiment of the first, second or third aspects of the invention, the defatting process can use other organic solvents instead of acetone, including but not limited to chloroform, hexane, heptanes or ethanol. The jack bean meal can be agitated in the acetone to allow for full dissolution of the fats. The agitation can be accomplished by any means known in the art, including by placing the jack bean meal/acetone mixture on a stir plate, as shown in FIG. 1. The jack bean meal/acetone mixture 13 in a beaker 12 can be placed on the stir plate 11. A magnetic stir bar 14 can be added to the beaker 12. The stir plate 11 can contain a magnet (not shown) that can spin, causing the magnetic stir bar 14 to spin and stir the jack bean meal/acetone mixture 13. The speed of the spinning can be controlled by dial 15. In any embodiment of the first, second or third aspects of the invention, the process of grinding the jack beans into jack bean meal may be omitted, and the whole, de-hulled jack beans can be defatted without grinding such that the purification of urease can be done on either whole beans or on jack bean meal. In any embodiment of the first, second or third aspects of the invention, the jack bean meal can be washed with hexane(s) and/or heptane(s) prior to the defatting with acetone in order to optimize fat removal prior to enzyme extraction.

In any embodiment of the first, second or third aspects of the invention, the jack bean meal/acetone mixture can be agitated for 30 minutes. In any embodiment of the first, second or third aspects of the invention, the jack bean meal/acetone mixture can be agitated for 15-29 minutes, 31-60 minutes, 60-120 minutes or even longer. The resulting mixture can then be filtered, and the remaining defatted jack bean meal kept.

In any embodiment of the first, second or third aspects of the invention, the defatting process can be repeated to ensure complete removal of the lipid material. In some embodiments, the agitation with cold acetone and filtering steps can be repeated 2, 3, 4, 5 or more times.

In any embodiment of the first, second or third aspects of the invention, the defatting process can use 750 mL of acetone for each 20 g of jack bean meal in each of the defatting steps. In any embodiment of the first, second or third aspects of the invention, any of 500 mL to 750 mL of acetone, 750 mL to 1 L of acetone, 1 L to 1.5 L of acetone, or more can be used for each 20 g of jack bean meal.

The filtering step of the defatting process can be accomplished with any known filtering means. In any embodiment of the first, second or third aspects of the invention, a glass filter can be used. The pore size of the filter must be small enough so that the urease containing jack bean meal does not pass through the filter. In any embodiment of the first, second or third aspects of the invention, the pore size of the filter can be 1.0 micron. In other embodiments, the pore size of the filter can be any of 0.2 to 0.7 micron, 0.7 micron to 1.0 micron, 1.0 micron to 1.2 micron, or 1.2 micron to 2.0 micron. One example of a filter that can be used is a Whatman GF/B Glass Microfibre Filter 12.5 cm, with a pore size of 1.0 micron, however any suitable filter is contemplated by this invention.

The urease can then be extracted from the defatted jack bean meal. In embodiment of the first, second or third aspects of the invention, a buffer at a pH of 7.2 can be added to the defatted jack bean meal. In any embodiment of the first, second or third aspects of the invention, a buffer at a pH between any of 7.0 to 7.2, 7.2 to 7.4, 7.4 to 7.7 or 7.7 to 8.0 can be added. In any embodiment of the first, second or third aspects of the invention, the buffer can be at any pH between 4.5 to 8.5. The jack bean meal can again be agitated in the buffer. In embodiment of the first, second or third aspects of the invention, the jack bean meal/buffer mixture can be cooled while agitating. In embodiment of the first, second or third aspects of the invention, the jack bean meal/buffer mixture can be cooled to a temperature between at or below 15° C. while agitating the mixture. In any embodiment of the first, second or third aspects of the invention, the agitation can take place in an ice bath. In any embodiment of the first, second or third aspects of the invention, the jack bean meal/buffer mixture can be agitated for 30 minutes. In any embodiment of the first, second or third aspects of the invention, the jack bean meal/buffer mixture can be agitated for 15-29 minutes, 31-60 minutes, 60-120 minutes or longer. The resulting jack bean meal/buffer mixture can then be filtered and the supernatant liquid can be retained.

The buffer used in the extraction of the first, second or third aspects of the invention can be any buffer known in the art usable with proteins and within the pH ranges above that will allow for the dissolution of urease. One example of a buffering solution that can be used is a mixture of 20 mM bis tris propane buffer pH 7.2 with 5 mM EDTA and 5 mM 2-mercaptoethanol (2-ME). In any embodiment of the first, second or third aspects of the invention, the buffer can be any buffer that functions in the pH range of 4.5 to 8.5 including phosphate, tris, MES, or others equivalent buffers known to those of ordinary skill in the art.

In any embodiment of the first, second or third aspects of the invention, the amount of buffer used can be critical. This is because the pH of jack bean meal samples in water varies depending on the source and age of the jack beans, as shown in Table 1. Pre-ground jack bean meal from Sigma in water was found to have a pH of 4.96, while fresh ground whole beans from Sigma was found to have a pH of 6.46. As the age of the jack beans increased, the pH of samples in water changed, such that 10 day old ground Sigma jack beans in water had a pH of 6.48, while 2 month ground Sigma jack beans in water had a pH of 5.86.

TABLE 1

| Sample Description | pH of Sample in Water |
|---|---|
| Sigma Pre-ground Meal | 4.96 |
| Fresh Ground Sigma Jack Beans | 6.46 |
| 10-Day Old Ground Sigma Jack Beans | 6.48 |
| 2-Month Old Ground Sigma Jack Beans | 5.86 |

Because the efficiency of the extraction process can be affected by the surface area of the jack bean meal, in any embodiment of the first, second or third aspects of the invention, the extraction can be carried out in a large vessel. For example, for 20 g of jack bean meal originally used, a 1000 mL vessel can be used for the extraction. In any embodiment of the first, second or third aspects of the invention, a larger or smaller vessel can be used. In embodiments of the first, second or third aspects of the invention where a small vessel is used for the extraction, it may require a longer agitation period to ensure full extraction of the urease from the jack bean meal.

In embodiment of the first, second or third aspects of the invention, the supernatant liquid obtained from filtering the buffered jack bean meal can be centrifuged. This is especially true if the supernatant liquid becomes turbid. The resulting liquid can be decanted and the supernatant liquid retained.

The urease from the original jack beans will be dissolved in the supernatant liquid. The urease can be purified through any means known in the art. In any embodiment of the first, second or third aspects of the invention, size exclusion chromatography can be used to purify the urease from the mixture, as shown in FIG. 2. In size exclusion chromatography, a column 21 can be filled with porous particles 22. Substances moving through the column may be able to enter the porous particles 22 or not, depending on their size. For example, a small molecule, represented by arrow 23 can enter the porous particles 22, while a larger molecule, represented by arrow 24, cannot. Therefore, the small molecule 23 will travel a further distance to move through the column than a larger molecule 24, because the smaller molecule can enter the porous particles 22. In any embodiment of the first, second or third aspects of the invention, ion exchange chromatography, hydrophobic interaction chromatography, or affinity chromatography can be used to purify the urease. It is contemplated that preparative size exclusion chromatography can be performed in any embodiment of the first, second or third aspects of the invention to purify the urease, and not simply as an analytical method. In any embodiment of the first, second or third aspects of the invention, the purified urease can be subjected to a lyophilization process or spray-drying to preserve the urease for future use.

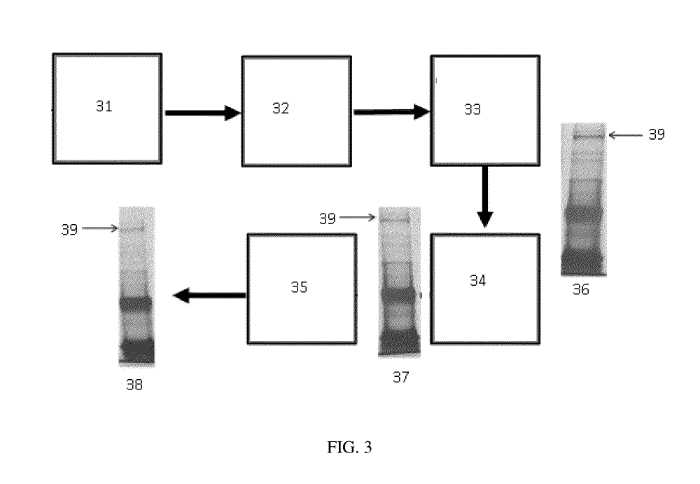

FIG. 3 shows a flow chart of the steps in the purification and extraction processes, with SDS-PAGE images showing the presence of urease and impurities in the mixtures. The jack beans can be shelled and ground to create jack bean meal 31. The jack bean meal can be defatted as described above 32. The urease can be extracted from the defatted jack bean meal with the buffer solution 33. SDS-PAGE image 36 was taken of the supernatant liquid after the urease has been extracted from the defatted jack bean meal 33. Band 39 shows the presence of the urease in the supernatant liquid. The supernatant liquid can be filtered as described above 34 to produce a liquid used for SDS-PAGE image 37. Again, the presence of urease is shown by band 39 in SDS-PAGE image 37. The extracted urease can then undergo the lyophilization process 35. After lyophilization 35, the presence of urease is shown in SDS-PAGE image 38 by band 39.

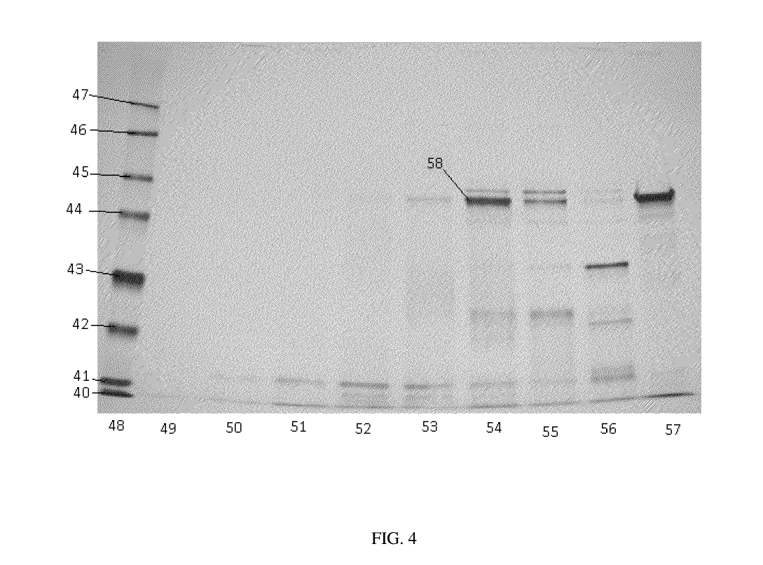

FIG. 4 shows a SDS-PAGE image obtained during the size exclusion chromatography of the extracted urease. The first column 48 is provided as a reference, wherein the band 40 corresponds to a molecular weight of 0 kD, band 41 corresponds to a molecular weight of 25 kD, band 42 corresponds to a molecular weight of 37 kD, band 43 corresponds to a molecular weight of 50 kD, band 44 corresponds to a molecular weight of 75 kD, band 46 corresponds to a molecular weight of 100 kD, band 47 corresponds to a molecular weight of 150 kD, and band 48 corresponds to a molecular weight of 250 kD. Each of the columns 49-56 correspond to fractions that eluted during size exclusion chromatography at different times. Column 57 is a control sample of urease. Band 58 shows the presence of urease. As can be seen by column 54, a fraction can be obtained wherein urease is the predominate species.

In any embodiment of the first, second or third aspects of the invention, the defatting process is not necessary. Instead, the urease can be extracted directly from the jack bean meal with an aqueous acetone solution that has not been cooled. Impurities within the extract can be precipitated out by alkalifying and heating the extract. In any embodiment of the first, second or third aspects of the invention, the pH of the extract can be raised to 8.5. This causes proteins with basic isoelectric points (i.e. >7.0, <8.6) to selectively precipitate. In any embodiment of the first, second or third aspects of the invention, the pH can be raised to any point between 7.5 and 9.0. In any embodiment of the first, second or third aspects of the invention, the urease can be precipitated from the impurities by acidifying the extract to just below the isoelectric point of urease, and heating the extract. In any embodiment of the first, second or third aspects of the invention, the pH of the extract can be lowered to 4.5. In any embodiment of the first, second or third aspects of the invention, the pH can be lowered to any point between 4.5 and 5.5. In any embodiment of the first, second or third aspects of the invention, the extraction can be carried out in the presence of 36% acetone. In any embodiment of the first, second or third aspects of the invention, the extraction is not carried out in the presence of acetone between 31 and 35%. In any embodiment of the first, second or third aspects of the invention, heating the extract can be omitted. The mixture in embodiments where base is added can undergo ultrafiltration to obtain a purified extract, with the supernatant liquid retained. In embodiments of the first, second or third aspects of the invention where acid is added, a solid material can be retained. The solvents can be removed from the urease to provide the purified urease. The urease can be further purified as described above, such as with size exclusion chromatography.

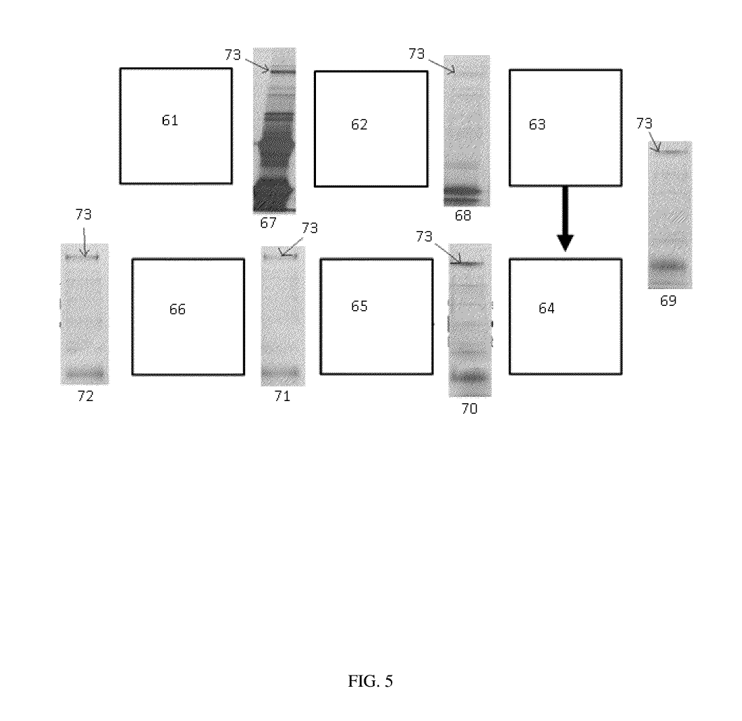

FIG. 5 shows a flow chart with SDS-PAGE images showing describing the purification and extraction of urease from jack beans without the defatting step. The jack beans can be first de-hulled and ground to make jack bean meal 61. An SDS-PAGE image of the jack bean meal 67 shows the presence of urease at band 73. The urease can be extracted from the jack bean meal with aqueous buffer 62. SDS-PAGE image 68 shows the presence of urease at band 73 in the extracted buffer solution. Impurities can be precipitated out 63 with base addition and heating as described above. SDS-PAGE image 69 shows the presence of urease at band 73 in the supernatant liquid after the precipitation. The solvent can be removed 64 to obtain a concentrated mixture. The mixture contains urease, as is shown by band 73 in SDS-PAGE image 70. The mixture can be diluted with buffer and ultrafiltered 65 to obtain the mixture in SDS-PAGE image 71, which contains urease as shown by band 73. The resulting urease can undergo a lyophilization process 66 to preserve the urease. SDS-PAGE image 72 taken of the lyophilized powder shows the presence of the urease at band 73.

In any embodiment of the first, second or third aspects of the invention, the urease can be purified by ultrafiltration as described herein. In any embodiment of the first, second or third aspects of the invention, the urease can be purified by diafiltration using an ultrafiltration membrane.

### Example 1

Fresh jack beans were de-hulled and ground to make jack bean meal. 20 g of jack bean meal were placed in a 1000 mL beaker, and 750 mL of ice cold acetone was added. The beaker was placed in an ice bath on a stir plate and stirred for 30 minutes. The resulting mixture was filtered using a Whatman GF/B Glass Microfibre Filter 12.5 cm, with a pore size of 1.0 micron. The acetone was discarded. The remaining jack bean meal was scraped into a 1000 mL beaker and 750 mL of ice cold acetone was again added. The mixture was again placed in an ice bath on a stir plate and stirred for 2 hours. The resulting mixture was filtered using the same filter and the acetone was discarded.

The remaining, now defatted, jack bean meal was scraped into a 1000 mL beaker, and 150 mL of cold 20 mM bis tris propane buffer pH 7.2 with 5 mM EDTA and 5 mM 2-mercaptoethanol (2-ME) was added. The ratio of jack bean meal to buffer is critical, as a less efficient extraction results when the amount of buffer is changed. Without being limited to any particular theory, it is believed residual acetone can be present in the defatted meal that ends up in the extraction buffer at some concentration. The beaker was placed on a stir plate in an ice bath and stirred for 30 minutes. The resulting mixture was filtered using a Whatman GF/B Glass Microfibre Filter 12.5 cm, with a pore size of 1.0 micron. The supernatant liquid was retained.

The resulting liquid, containing the urease, reduced to 0.3 mL under vacuum. Water was added to make the total volume 1.5 mL. 1.0 mL of this liquid was purified by size exclusion chromatography (˜150 mL of Superdex® 200, prep grade, GE Healthcare) to obtain the purified urease.

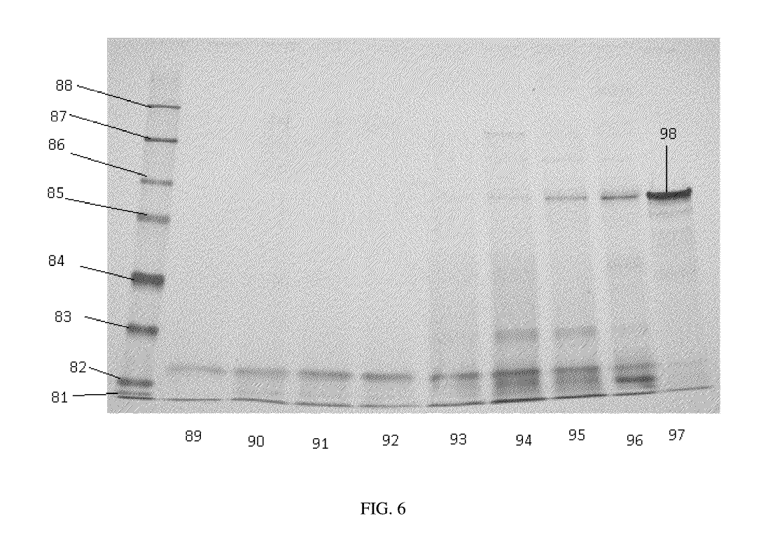

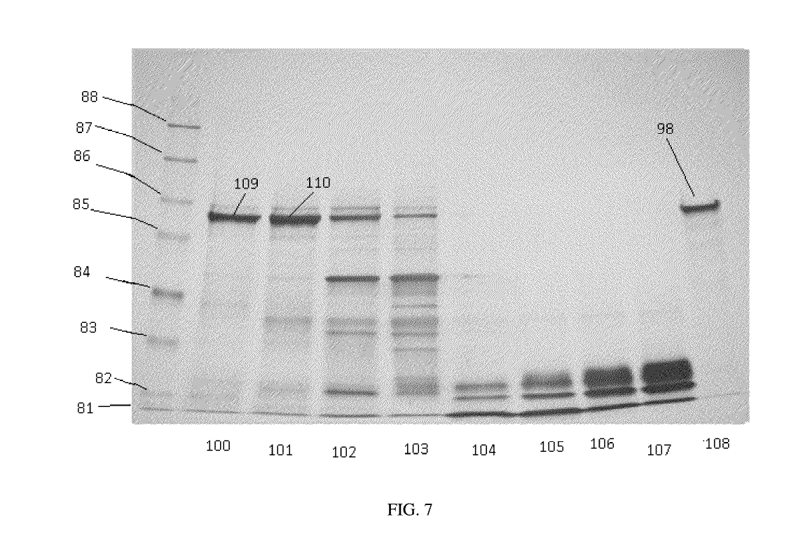

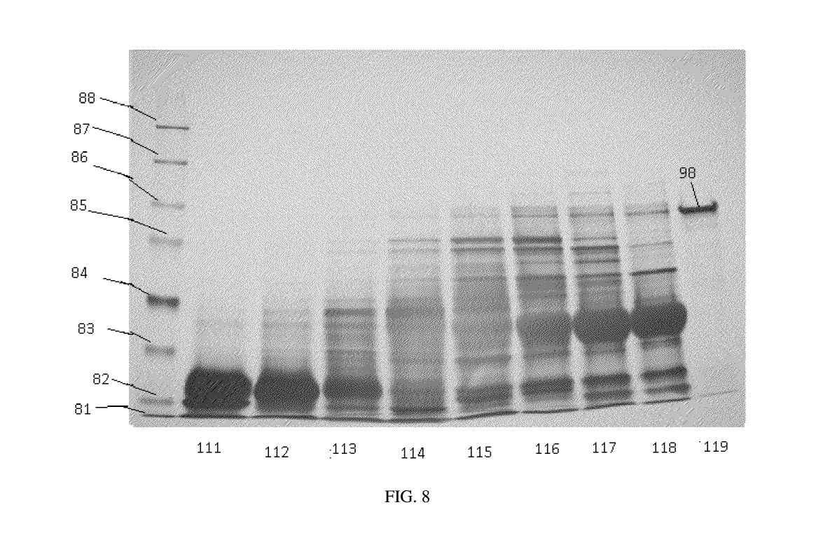

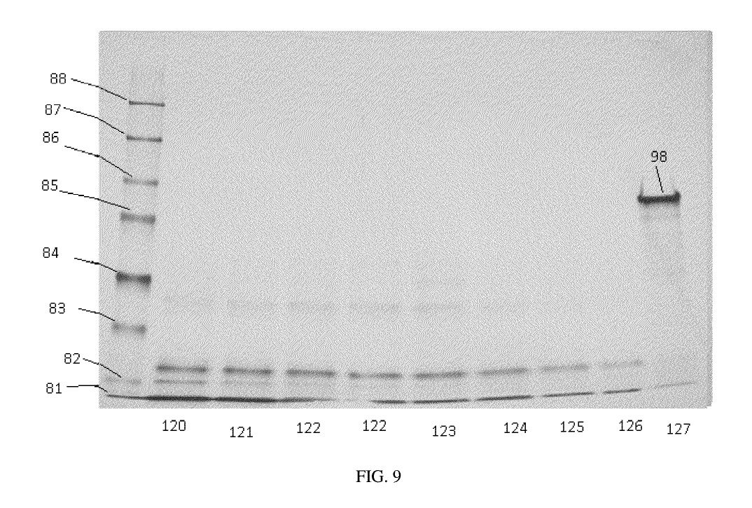

FIGS. 6-9 show SDS-PAGE images of the fractions collected during the size exclusion chromatography. FIG. 6 shows the first 8 fractions collected 89-96, FIG. 7 shows the next 8 fractions collected 100-107, FIG. 8 shows the next 8 fractions collected 111-118, and FIG. 9 shows the last 8 fractions collected 120-126. For each of FIGS. 6-9, band 81 corresponds to a molecular weight of 0 kD, band 82 corresponds to a molecular weight of 25 kD, band 83 corresponds to a molecular weight of 37 kD, band 84 corresponds to a molecular weight of 50 kD, band 85 corresponds to a molecular weight of 75 kD, band 86 corresponds to a molecular weight of 100 kD, band 87 corresponds to a molecular weight of 150 kD, and band 88 corresponds to a molecular weight of 250 kD. A reference sample of urease is provided in column 97 of FIG. 6, column 108 of FIG. 7, column 119 of FIG. 8, and column 127 of FIG. 9, showing urease at band 98. The volume of each fraction is 4 mL.

As can be seen, the majority of the urease was collected in fractions 100 and 101, as shown by urease bands 109 and 110 in FIG. 7. In the size exclusion chromatography depicted, 1707 international units were loaded on to the column. In fractions 100 and 101, 1140 international units were recovered. This corresponds to a yield of about 67% for the size exclusion chromatography.

### Example 2

The reagents used for the urease extraction and purification were 100 mM phosphate buffer, pH 7.0; 15 mM phosphate buffer, pH 7.0; 100 mM 2-mercaptoethanol; 100 mM EDTA; ACS grade acetone, 2 M sodium hydroxide and jack bean meal from de-hulled beans.

Fresh jack beans were de-hulled and ground to make jack bean meal. 50 g of jack bean meal were placed in a 1000 mL beaker. To this was added, drop-wise, 170 mL of 100 mM phosphate buffer, 80 mL of acetone and 2.5 mL each of EDTA and 2-mercaptoethanol to make a solution 32% acetone. The mixture was stirred for 15 minutes, and then centrifuged at 4,000-6,000 g for 15 minutes while keeping the mixture at 20° C. The liquid was then decanted and filtered through a 3 micron filter.

The extract was transferred to a 250 mL Erlenmeyer flask. 13 mL of acetone was added drop-wise. The pH of the extract was raised to 8.5 using 2.5 M NaOH, creating a 36% acetone solution. The mixture was then heated to 40-42° C. for 20 minutes. The mixture was cooled to room temperature and centrifuged at 4,000-6,000 g for 15 min. The liquid was again decanted.

The solvent was removed using a rotovap at 32° C. water bath, beginning at 250 mBar, and reducing vacuum to 20 mBar over approximately 45-60 min. It was then filtered through a 25 micron 0.22 μm membrane filter.

The sample was next diluted to 300 mL with a 15 mM phosphate buffer. An ultrafiltration cell was assembled with a 100 kDa cellulose membrane. The diluted sample feed was added to the cell, and the volume recorded. The pressure cap on the ultrafiltration cell was replaced, and the cell was placed on a magnetic stirrer in a refrigerator at 5° C. The pressure regulator was backed off so that there was no flow while the nitrogen was turned on. The nitrogen was turned on, and the pressure slowly increased. The cell was stirred at a stirring level of 4. An outlet tube was placed into a suitable reservoir to catch the filtrate. The volume of the cell was monitored. When the cell volume reached 50 mL, the pressure was released and the cell contents diluted back to 300 mL using 15 mM phosphate buffer. The nitrogen was again turned on, the cell stirred, and the ultrafiltrate collected until the cell again reached 50 mL. The nitrogen was shut off, the pressure was vented and the cell decanted into a cylinder. The retentate was stored at 3-5° C.

### Example 3

750 mL of acetone was added to 20 g of jack bean meal and the mixture stirred. The mixture was filtered with a Whatman GF/B Glass Microfibre Filter 12.5 cm, pore size 1.0 micron. 750 mL of acetone was added to the solid material, and again was filtered using a Whatman GF/B Glass Microfibre Filter 12.5 cm, pore size 1.0 micron. Immediately after filtering, the defatted jack bean meal was subjected to a cold buffer extraction using 150 mL of 20 mM bis tris propane buffer pH 7.2. The ratio of jack bean meal to buffer is critical, as a less efficient extraction results when the amount of buffer is changed. Without being limited to any particular theory, it is believed that residual acetone in the defatted meal can be present in the extraction buffer at some concentration. The mixture was filtered using a Whatman GF/B Glass Microfibre Filter 12.5 cm, pore size 1.0 micron. The extract was centrifuged at 2500 rpm at 4° C. for 30 minutes. The supernatant liquid was decanted and the pellets discarded. The pellets are devoid of any urease activity. A SFCA (0.2 μm) filter was used to filter the supernatant liquid. A speed vac was used on 5 mL of the buffer extract. The total volume recovered was 0.3 mL, which was diluted to 1.5 mL with water. Using a Wyatt switching valve, 1 mL of the 1.5 mL solution was loaded onto a pre-equilibrated preparative SEC column. A fraction collector was used to collect 4 mL fractions starting at 80 minutes.

### Example 4

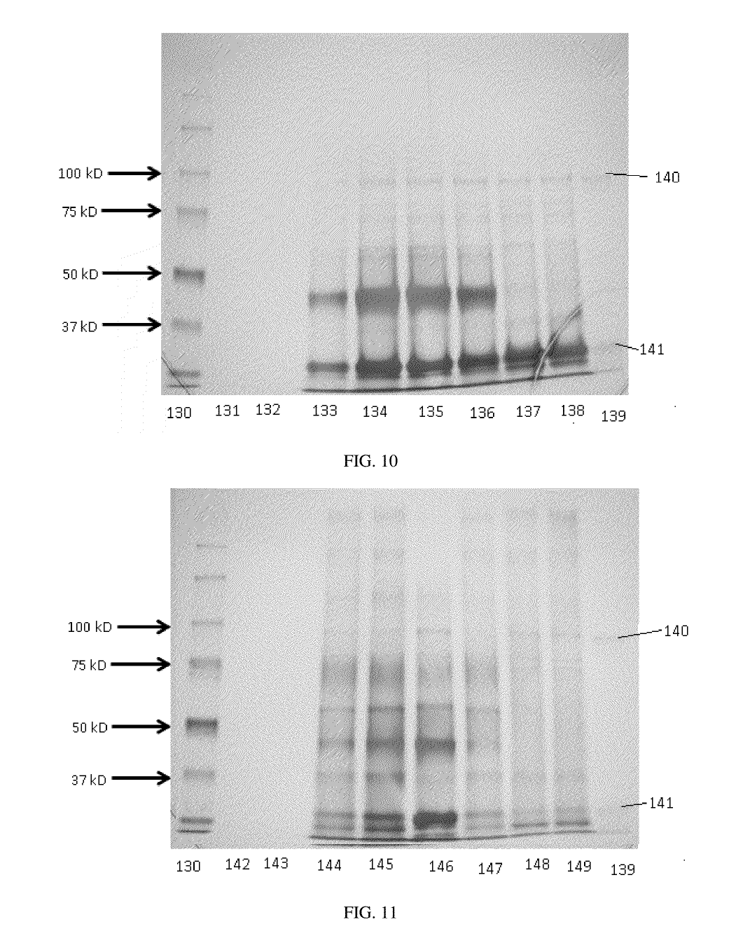

To determine the effect of temperature at different points in the purification method, various conditions were tested, as shown in Table 2. FIG. 10 shows an SDS-PAGE under reducing conditions, while FIG. 11 shows an SDS-PAGE under non reducing conditions. Each of the samples in Table 2 shows reference numerals from FIGS. 10 and 11 corresponding to the column the sample appears in. The first reference numeral corresponds to the column in which the sample appears in FIG. 10, and the second reference numeral corresponds to the column in which the sample appears in FIG. 11. In both FIGS. 10 and 11, column 139 is a standard containing Con A, shown by the band at 141, which corresponds to a molecular weight near 25 kDa, and urease shown by the band at 140. In both FIGS. 10 and 11, column 130 shows standardized weights for reference to the bands in the image. BTP stands for bis tris propane buffer. SDS stands for sodium dodecyl sulfate. UF stands for ultrafiltration. Con A stands for concancavalin A.

TABLE 2

| | | Reference |
|---|---|---|
| | | Number in |
| Step | Conditions | FIG. 10, 11 |
| Acetone Wash | Room temperature, stirred for | 131, 142 |
| | 20 minutes | |
| Acetone Wash | Ice cold, stirred for 2 hours | 132, 143 |
| Buffer Extraction dJBM | Room temperature, stirred for | 133, 144 |
| | 30 minutes | |
| Buffer Exchange dJBM | BTP with 2% SDS, UF | 134, 145 |
| Buffer Exchange dJBM | BTP with 2% SDS, UF, | 135, 146 |
| | supernatant | |
| Buffer Exchange dJBM | BTP, UF | 136, 147 |
| Buffer Extract dJBM | Ice cold BTP with protease | 137 148 |
| | inhibitors | |
| Buffer Extract dJBM | Ice cold BTP | 138, 149 |
| Urease and Con A | N/A | 139 |
| standard | | |

The SDS PAGE was run under both reducing conditions, as shown in FIG. 10 and non-reducing conditions, as shown in FIG. 11, in order to characterize the multimeric form of proteins due to disulfide bridge formation. The disulfide bridges are reduced under the reducing conditions so that any multimers present would be broken down into the monomer form. The results are consistent across both sets of conditions.

As can be seen from FIGS. 10 and 11, the step of defatting the jack bean meal with acetone did not result in any loss of urease. This is shown by a lack of urease activity in columns 131 and 132 in FIG. 10 and columns 142 and 143 in FIG. 11.

The temperature of the extraction was significant. As shown in FIGS. 10 and 11, a room temperature extraction resulted in an impurity at around 45 kDa, as seen in column 133 in FIG. 10 and column 144 in FIG. 11. By contrast, when cold buffer was used for the extraction, as shown in column 138 in FIG. 10 and column 149 in FIG. 11, very little of the impurity was present. The impurity at 45 kDa was present in all runs that used room temperature buffer, as shown in columns 133-136 in FIG. 10 and columns 144-147 in FIG. 11.

Con A, which is a lecithin carbohydrate binding protein, was added to the standard because it is known to be a protein present in jack beans. It is possible that the impurities at low molecular weight include Con A, but this has not been conclusively shown. It is noted that Con A can be separated from urease using size exclusion chromatography.

In some experiments, SDS was added in order to break up aggregated urease into the monomeric form prior to ultrafiltration. The addition of SDS did not significantly change the levels of impurities. This can be seen by comparing column 134 with column 136 in FIG. 10 and column 145 with column 147 in FIG. 11. Likewise, the addition of protease inhibitors, which were added to prevent the break-down of urease by proteases in the jack bean meal, did not significantly change the results, as can be seen by comparing column 137 to column 138 in FIG. 10 and column 148 to column 149 in FIG. 11.

One skilled in the art will understand that various combinations and/or modifications and variations can be made in the dialysis system depending upon the specific needs for operation. Moreover, features illustrated or described as being part of an aspect of the invention can be included in the aspect of the invention, either alone or in combination.

We claim:

1. A method for isolating and purifying urease from natural sources of urease comprising the steps of:
defatting the natural sources of urease using cold solvent to create defatted natural sources of urease;
extracting urease from the defatted natural sources of urease; and
purifying the resulting urease solution.

2. The method of claim 1 wherein the natural source of urease is jack beans.

3. The method of claim 2, wherein the hulls of the jack beans are removed prior to the step of defatting the jack beans.

4. The method of claim 3, wherein the jack beans are ground to make jack bean meal prior to the step of defatting the jack beans.

5. The method of claim 1, wherein the step of defatting the natural sources of urease comprises mixing the natural sources of urease with cold solvent to create a natural sources of urease/cold solvent mixture, and mechanically separating the natural sources of urease/cold solvent mixture.

6. The method of claim 1, wherein the step of extracting urease from the defatted natural sources of urease comprises:
adding the defatted natural sources of urease to a buffer solution to create a natural sources of urease/buffer mixture;
agitating the natural sources of urease/buffer mixture; and
mechanically separating the natural sources of urease/buffer mixture wherein the supernatant liquid contains the urease.

7. The method of claim 6, wherein the buffer solution is a cold aqueous buffer.

8. The method of claim 1, wherein the step of purifying the urease solution comprises using any one of size exclusion chromatography, hydrophobic interaction chromatography, affinity chromatography, or ultrafiltration to purify the urease.

9. The method of claim 1 wherein the cold solvent is ice cold solvent; wherein the ice cold solvent is kept at a temperature of between −5° C. and 5° C.

10. The method of claim 1 wherein the cold solvent is cold acetone.

11. The method of claim 1, further comprising the step of subjecting the purified urease to any one or both of a lyophilization process or spray-drying.

12. A method for isolating and purifying urease from natural sources of urease comprising the steps of:
extracting the urease from the natural sources of urease with acetone and buffer to make an extracted urease solution;
precipitating out impurities from the extracted urease solution;
mechanically separating the extracted urease solution to remove the precipitated impurities; and
purifying the urease solution.

13. The method of claim 12 wherein the step of precipitating out impurities from the extracted urease solution comprises:
adding base to the extracted urease solution; and
heating the extracted urease solution.

14. The method of claim 12 wherein the step of purifying the urease comprises removing the acetone and buffer from the urease;
dissolving the remaining material in water to make an aqueous solution; and
mechanically separating the aqueous solution to obtain a supernatant liquid, wherein the urease is present in the supernatant liquid.

15. The method of claim 14 wherein the step of purifying the urease solution further comprises using any one of size exclusion chromatography, hydrophobic interaction chromatography, affinity chromatography and ultrafiltration to purify the urease solution.

16. The method of claim 12 wherein the acetone and buffer are cold acetone and buffer.

17. The method of claim 12 further comprising the step of defatting the natural sources of urease prior to extracting the urease.

18. The method of claim 1 further comprising the step of washing the natural sources of urease with hexane and/or heptane prior to the step of defatting the natural sources of urease.

19. A method of purifying urease, comprising the step of: modulating the pH of a liquid in the presence of urease.

20. The method of claim 19 further comprising the step of mechanically separating fractions after modulating the pH of the liquid to isolate the fractions having an enhanced urease activity, wherein the urease activity of each fraction is defined on a per unit volume, dry mass, or protein mass basis.
